(19) United States
(12) Patent Application Publication
(10) Pub. No.: US 2004/0058309 A1
(43) Pub. Date: Mar. 25, 2004

(54) Plasma supplement and use in liver assist systems

(76) Inventors: Junji Washizu, Kasugai (JP); Mehmet Toner, Wellesley, MA (US); Martin L. Yarmush, Newton, MA (US); Francois Berthiaume, Boston, MA (US)

Correspondence Address: FISH & RICHARDSON PC, 225 FRANKLIN ST, BOSTON, MA, 02110, US

(21) Appl. No.: 10/398,995

(22) PCT Filed: Oct. 12, 2001
(86) PCT No.: PCT/US01/32052

(51) Int. Cl. A01N 1/02; A61K 38/28; A61K 31/405; A61K 31/198; A61K 31/573

(52) U.S. Cl. 435/2; 514/3; 514/171; 514/419; 514/561

(57) ABSTRACT

The invention relates to a plasma or blood supplement to be added to plasma or blood during clinical use of liver assist systems.

### TECHNICAL FIELD

This invention relates to the culturing of cells in plasma for use in liver assist systems.

### BACKGROUND

Bioartificial liver assist systems are a promising avenue for treating liver failure due to their potential to express a full range of metabolic, synthetic, and detoxifying functions. Since hepatocytes are the major component of such systems, considerable effort has been expended towards establishing an environment where these cells can maintain long-term viability and differentiated function in vitro. Recent successes include the development of collagen gel sandwich culture systems for primary hepatocytes, co-culture with epithelial, sinusoidal, or mesenchymal cells, culture as spheroids in collagen, or culture on extracellular matrix extracted from Engelbroth-Holm-Swarm sarcoma. While such systems perform well when used with culture medium, they typically fail when used with plasma, the true culture medium during clinical usage in that hepatocellular function significantly decreases.

### SUMMARY

The invention is based on the discovery that by supplementing plasma used in liver assist systems during clinical use with specific concentrations of certain amino acids and hormones, the function of hepatocytes growing in the system can be significantly enhanced and prolonged.

In general, the invention features a plasma or blood supplement to be mixed with plasma or blood, the supplement including amounts of insulin, glycogen, hydrocortisone, and one or more amino acids sufficient to provide a concentration in plasma or blood of: from 50 μU/mL to 1000 mU/mL insulin; from 0.7 ng/mL to 28 ng/mL glucagon; from 75 ng/mL to 15 μg/mL hydrocortisone; and from 5.3 to 27.6 mM of one or more amino acids.

The supplement can provide a concentration in plasma (or blood) of 2.0 mM-10.0 mM of L-glutamine, and can further provide a concentration of: 0.1 mM-0.8 mM of L-histidine, 0.4 mM-2.0 mM of L-isoleucine, 0.4 mM-2.0 mM of L-leucine, 0.4 mM-2.0 mM of L-lysine, 0.1 mM-0.8 mM of L-methionine, 0.2 mM-1.0 mM of L-phenylalanine, 0.4 mM-2.0 mM of L-threonine, 0.04 mM-0.2 mM of L-tryptophan, and 0.4 mM-2.0 mM of L-valine.

The supplement can further include a calcium salt, e.g., calcium chloride, in an amount sufficient to provide a concentration of from 1.8 to 12.8 mM of the calcium salt, and a magnesium salt, e.g., magnesium sulfate, in an amount sufficient to provide a concentration of from 0.8 to 5.7 mM of the magnesium salt.

The invention also features a method of enhancing hepatocyte viability and function in a liver assist system (LAS) during clinical use by culturing hepatocytes in the LAS using standard culture medium; after the hepatocytes are established in the LAS, removing the culture medium and adding plasma or blood; and adding to the LAS the plasma or blood supplement of the invention in an amount effective to enhance the viability and function of the hepatocytes.

The invention provides several advantages. In particular, the new methods and compositions allow for the simple supplementation of liver assist systems, e.g., at the plasma inlet port, to significantly enhance the metabolic function and longevity of the hepatocytes. These cells would otherwise quickly lose their function when exposed to plasma under clinical conditions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A and 1B are graphs showing the effect of sodium chloride concentration in supplemented plasma. Asterisks (*) indicate a significant difference from cultures with full sodium chloride (110 mM) supplementation.

FIGS. 2A and 2B are graphs showing the role of inorganic salts (Fe, Mg, K, and Ca) in plasma supplementation. Asterisks (*) indicate a significant difference from cultures in plasma with 5× concentration of all inorganic salts.

FIGS. 3A and 3B are graphs showing the role of amino acids (complete amino acid mixture from Basal Medium Eagle (BME), Gln, Gly, None) as supplements to plasma. Asterisks (*) indicate a significant difference from cultures in plasma supplemented with BME amino acids.

FIGS. 4A and 4B are graphs showing the role of the hormones insulin, glucagon, and hydrocortisone in urea and albumin secretion during plasma exposure. Asterisks (*) indicate a significant difference from cultures in plasma supplemented with all three hormones.

FIGS. 5A and 5B are graphs showing liver-specific functions of hepatocytes during exposure to heparin-treated plasma. Albumin synthesis rate (5A) and urea synthesis rate (5B) are shown for cells cultured in standard culture medium (M), or exposed to non-supplemented (P), hormone supplemented (P+H), or hormone plus amino acid supplemented (P+H+A) plasma. Data are shown as the average±SD of three independent experiments, each using triplicate cultures. Asterisks (*) indicate a significant difference (p<0.05) from standard culture medium cultures.

FIGS. 6A and B are graphs showing cytochrome P450 activities of hepatocytes co-cultured with fibroblasts during exposure to heparin treated plasma. Ethoxyresorufin de-ethylase activity (EROD-6A) and pentoxyresorufin de-ethylase activity (PROD-6B) are shown for cells cultured in standard culture medium (DMEM), standard culture medium with 2 μM methylcholanthrene (DMEM+3MC) or 1 mM phenobarbital (DMEM+PB) added as P450 inducers, or exposed to non-supplemented plasma with P450 inducers (plasma+3MC or plasma+PB), or hormone plus amino acid supplemented plasma (plasma+3MC+hor+aa or plasma+PB+hor+aa). Data are shown as the average±SD of three independent experiments, each using triplicate cultures. Asterisks (*) indicate a significant difference (p<0.05) from standard culture medium cultures.

FIGS. 7A and 7B are graphs showing liver-specific functions of hepatocytes co-cultured with fibroblasts during exposure to heparin-treated plasma. Urea synthesis rate (7A) and albumin synthesis rate (7B) are shown for cells cultured in standard culture medium (DMEM), or exposed to non-supplemented (plasma), or hormone plus amino acid supplemented (plasma+sup) plasma. Data are shown as the average±SD of two independent experiments, each using triplicate cultures. Asterisks (*) indicate a significant difference (p<0.05) from standard culture medium cultures.

### DETAILED DESCRIPTION

The invention is based on the discovery that by supplementing plasma used in liver assist systems during actual clinical use with specific concentrations of certain amino acids, hormones, and/or cations, the function of hepatocytes maintained in the liver assist system can be significantly enhanced and prolonged.

Maintenance of liver-specific functions in hepatocyte cultures during plasma exposure is critically important for the successful clinical application of bioartificial liver assist systems. Hepatocytes exposed to citrated human plasma quickly lose urea and albumin synthesis, which is explained in part by a decrease in viability. As shown in the examples below, supplementing plasma with a concentrated supplement restores viability and hepatocyte-specific functions throughout a one-week exposure period. The supplement components necessary for maintaining urea synthesis include the inorganic salts calcium and magnesium, certain amino acids, and the hormone glucagon. Where albumin synthesis is also to be maintained, the plasma supplement additionally should include the hormones hydrocortisone and insulin.

In current bioartificial liver assist systems, blood or plasma carries toxins from the patient to the hepatocytes and hepatocyte-derived products back to the patient. See, e.g., Yarmush et al., Cell Transplant, 1:323 (1992), and Arkadopoulos et al., Int'l J. Artif. Organs, 21:781 (1998). Blood or plasma is perfused through an extracorporeal circuit, thus requiring an anticoagulant agent to avoid clotting. Citrate has been extensively used in hemodialysis as well as for blood transfusion, and its safety has been established in continuous procedures. Despite the fact that citrate has already been used in some clinical trials involving bioartificial liver assist systems, the toxicity of citrated plasma on cultured hepatocytes has been previously reported (Cunningham et al., Int'l J. Artif. Organs, 15:162, 1992), which raises concerns as to the viability and function of the hepatocytes in the device. In fact, most of the cell culture and animal studies to date have employed heparin as the anticoagulant.

Although heparin has not been reported to be directly toxic to hepatocytes, it is known to activate lipoprotein lipase and induce expression of hepatic lipase. Test results have shown that the resulting elevation in free fatty acid content in heparinized plasma induces massive intracellular lipid deposition (Matthew et al., Biotech. Bioeng., 51:100, 1996). As demonstrated below, the addition of hormones and amino acids to heparinized human plasma allows hepatocytes to maintain urea and albumin synthesis during a 7-day plasma exposure, and prevents lipid accumulation. Thus, the new methods and plasma supplements work for both citrated and heparinized plasma, although cations, such calcium and magnesium, are not required for use with heparinized plasma.

Patients undergoing liver failure are likely to have coagulation insufficiency due to liver failure. Because regional heparin anticoagulation is technically more difficult to control than regional citrate anticoagulation, heparin has the potential to increase the risk of hemorrhage in the patient. Thus, citrate anticoagulation, when used in conjunction with the supplementation described herein, may be preferable to heparin anticoagulation during bioartificial liver treatment. Either way, the new plasma supplement is effective to enhance the viability and function of hepatocytes.

Hepatocytes can also be co-cultured with other cells such as fibroblasts, endothelial cells, and nonparenchymal liver epithelial cells, providing a “feeder layer” in liver assist systems. Cultures must be maintained in medium for about one week prior to plasma exposure otherwise nonparenchymal cells tend to detach from the culture substrate. After this period, plasma does not affect these cells. As in the case of pure hepatocyte cultures, use of the new plasma supplement is necessary to maintain viability and function of the hepatocytes during subsequent plasma exposure.

Components of the Plasma Supplement

The components of the plasma supplement for maintaining hepatocyte-specific functions and viability in plasma are certain amino acids and certain hormones. In the case of citrated plasma, divalent cations, such as calcium and magnesium added as inorganic salts, are also required.

Inorganic salts are important for use with citrated plasma for the following reasons. Sodium citrate chelates divalent cations such as calcium and magnesium, thus decreasing free cations available for the hepatocytes. Extracellular calcium is important in maintaining hepatocyte plasma membrane integrity. Based on the formation constants for citrate and metal complexes (log K=4.68 for calcium, 3.29 for magnesium) the free calcium and magnesium concentrations in citrated plasma (using standard citrate dosages) should be in the micromolar range. Thus, supplementation with 1.8 mM calcium and 0.8 mM magnesium would increase the estimated free cation availability by 10-fold or less, which is sufficient to support hepatocyte-specific functions without inducing fibrin formation. The threshold free calcium level for thrombin activation is 0.25 mM, which cap be reached by increasing the total calcium concentration to 12.8 mM. Thus, the calcium supplementation used is not likely to increase the risk of clotting in an extracorporeal circuit.

The useful range of calcium ion supplementation in the plasma during clinical use in a liver assist system is therefore 1.8 to 12.8 mM, e.g., 1.8 or 2.5 to 6.4 mM, which is added during use of a standard liver assist system in the form of, e.g., calcium chloride, or any other soluble calcium salt.

Based on similar reasoning, the useful range of magnesium ion supplementation in the plasma during clinical use in a liver assist system is 0.8 to 5.7 mM, e.g., 0.8 or 1.3 to 2.8 mM, which is added during use of a standard liver assist system in the form of, e.g., magnesium sulfate, or any other soluble magnesium salt

Calcium and magnesium supplementation offsets chelation by citrate; thus the supplementation should be adjusted if the amount of citrate added to the plasma is changed so that the final concentration in plasma is in the micromolar range. The amount of supplement should be determined as outlined in the prior paragraph.

Amino acids are important for use in the plasma supplement to enhance hepatocellular protective mechanisms against cytotoxic factors such as complements activated by naturally occurring antibodies and protease-mediated destruction of the surrounding extracellular matrix. Certain amino acids such as glycine and alanine protect cells from damage caused by energy depletion, e.g., caused by anoxia or inhibition of mitochondrial activity. Glycine, cysteine, and glutamine are precursors of, and regulate the synthesis of, glutathione, a natural anti-oxidant that protects cells by detoxifying hydrogen peroxide produced during peroxidation of unsaturated free fatty acids or through xenobiotic metabolism.

In addition, synthesis of liver-specific proteins such as albumin requires a full complement of amino acids and the removal of even a single amino acid may have deleterious effects on this aspect of hepatic function. Thus, a full complement of amino acids is preferred. The total amino acid component of the plasma supplement amounts to about 10.7 mM (final concentration in plasma), which is not expected to have deleterious osmotic effects. The supplementation should not exceed about 25 mM of amino acids.

The useful range of most of the amino acids to be used in the plasma during clinical use of a liver assist system is from about 0.1 to 0.4 up to about 0.8 to 2.0 or even 10.0 mM depending on the amino acid (see Table 1 below). Thus, a plasma supplement to be added during use of a standard liver assist system should increase total plasma amino acid levels by 5.3 to 27.6 mM. The useful range of supplementation for each individual amino acid is shown in Table 1.

TABLE 1
Amino Acids for Use in Plasma Supplement

| | Ideal Plasma | | Useful |
|---|---|---|---|
| Amino Acid | Concentration | | Range |
| Glycine (0.8 M) | 0.4 | mM | 0.2 mM-1.0 mM |
| L-serine (0.8 M) | 0.4 | mM | 0.2 mM-1.0 mM |
| L-glutamine (0.2 M) | 4.0 | mM | 2.0 mM-10.0 mM |
| L-arginine (5 mM) | 0.4 | mM | 0.2 mM-1.0 mM |
| L-cystine (2.5 mM) | 0.2 | mM | 0.1 mM-0.8 mM |
| L-histidine (2.6 mM) | 0.2 | mM | 0.1 mM-0.8 mM |
| L-isoleucine (10 mM) | 0.8 | mM | 0.4 mM-2.0 mM |
| L-leucine (10 mM) | 0.8 | mM | 0.4 mM-2.0 mM |
| L-lysine (10 mM) | 0.8 | mM | 0.4 mM-2.0 mM |
| L-methionine (2.5 mM) | 0.2 | mM | 0.1 mM-0.8 mM |
| L-phenylalanine (5 mM) | 0.4 | mM | 0.2 mM-1.0 mM |
| L-threonine (10 mM) | 0.8 | mM | 0.4 mM-2.0 mM |
| L-tryptophan (1 mM) | 0.08 | mM | 0.04 mM-0.2 mM |
| L-tyrosine (5 mM) | 0.4 | mM | 0.2 mM-1.0 mM |
| L-valine (10 mM) | 0.8 | mM | 0.4 mM-2.0 mM |

Of the amino acids listed in Table 1, glutamine (e.g., at 10 mM final concentration in plasma) is essential to maintain cell viability and urea synthesis, the latter of which is important for ammonia detoxification. To support protein synthesis, nutritionally essential amino acids, i.e. L-histidine, L-isoleucine, L-leucine, L-lysine, L-methionine, L-phenylalanine, L-threonine, L-tryptophan, and L-valine should also be included. The addition of the other amino acids, i.e., glycine, L-serine, L-arginine, L-cystine, and L-tyrosine, reduces the need for cells to synthesize them intracellularly, which increases the efficiency of cellular metabolism. A combination of all of the amino acids in Table 1 is preferred.

Certain hormones are also to be included in the plasma supplement. In particular, three hormones, hydrocortisone, insulin, and/or glucagon, are also shown herein to be essential to maintain hepatocyte viability, morphology, and function. Based on the examples described herein, plasma supplemented with hydrocortisone; and insulin maintains hepatocyte morphology for at least one week in collagen gel sandwich devices, although hydrocortisone supplementation alone had no effect on urea synthesis (suggesting that plasma levels of this hormone were sufficient for urea synthesis). Glucagon and glucocorticoids coordinately induce enzymes in the urea cycle, which is consistent with the finding that glucagon supplementation was necessary for maintaining urea synthesis in plasma culture.

Although a single hormone may be sufficient to restore a particular function, all three are needed at the same time to induce hepatocytes to express the full complement of functions required in the liver assist device. The preferred and useful levels of hormones in the plasma supplement are indicated in Table 2 below. The values shown are plasma concentrations provided after supplementation.

TABLE 2
Hormones for Use in Plasma Supplement

| | Ideal Plasma | | Useful |
|---|---|---|---|
| Hormone | Concentration | | Range |
| Insulin (100 U/mL) | 500 | mU/mL | 50 μU/mL-1000 mU/mL |
| Glucagon (10 μg/mL) | 14 | ng/mL | 0.7 ng/mL-28 ng/mL |
| Hydrocortisone | 7.5 | μg/mL | 75 ng/mL-15 μg/mL |
| (2 mg/mL) | | | |

Preparation of the Plasma Supplement

The components of the supplement are obtained in pure form, or can be purified and sterilized prior to mixing. In one embodiment, the components are mixed together in purified, sterilized water and used immediately or lyophilized to prepare a powder for long-term storage in a cool, dry place. In another embodiment, the plasma supplement is prepared by mixing together the appropriate amounts of amino acids and salts in solid anhydrous form and lyophilized hormone powders. In either case, the powder is reconstituted with sterile water. If using nonsterile water, then the mixture would need to be sterilized, e.g., filter-sterilized through a 0.2 μm filter. Once reconstituted, the supplement should be stored at 4° C. and used within 24 hours or so. Alternatively, all the components except the hormones and glutamine could be stored in sterile solution under refrigeration. At time of use, glutamine and hormones would be added to the stored solution. The final concentration of the components in the supplements should be 5 to 20 times the concentration desired in the plasma. Thus, plasma and supplement are mixed with a 4:1 to 19:1 volume ratio.

Use of the Plasma Supplement

For clinical use in a bioartificial liver assist system, the plasma supplement is added continuously to the plasma before it enters the liver assist device of the system. The flow rate of the plasma supplement is adjusted to achieve the proper plasma levels of supplement. In general, the flows of plasma and supplement are controlled by separate peristaltic pumps, either manually or using a computer-based controller. For example, if the plasma supplement contains 5 times the desired plasma concentration, 1 part of supplement is mixed with 4 parts of plasma. The ratio depends on the concentration of the supplement. The source of plasma would be either arterial or venous blood. Blood is withdrawn continuously from the patient by a peristaltic pump, which sends it into a plasma separator. A second pump controls the flow rate at which the plasma comes out of the plasma separator, which may not exceed about half of the blood flow rate. A third pump continuously adds the supplement to the plasma line, e.g., via a “T” connection. For example, if the plasma supplement contains 5 times the desired plasma concentration, 1 part of supplement must be mixed with 4 parts of plasma; thus the flow rate of supplement is adjusted to ¼ the flow rate of plasma. The pumps can be controlled manually or by computer.

Plasma levels of metabolites should be monitored every 4 to 6 hours and the supplementation is adjusted accordingly so that metabolites in the plasma entering the device remain in the useful range. Frequent monitoring would also be useful to assess the progression of hepatic disease and effectiveness of bioartificial liver treatment. For example, once the patient's plasma metabolite levels returned to the normal range, bioartificial liver treatment would be discontinued.

Methods of Determining Hepatocyte Morphology and Function

While morphology can be determined microscopically, the expression of two major liver-specific functions, urea and albumin synthesis, are suitable indicators of hepatocyte metabolic function. These functions are altered extensively during plasma exposure since plasma obtained from peripheral blood differs from portal vein blood to which hepatocytes are exposed in vivo, especially in the postprandial state, when portal blood carries higher levels of nutrients derived from the gut. In a detailed study with controlled protein feeding in rats, the concentration of amino acids in the portal vein increased from a basal level of approximately 3 mM to 7.5 mM after protein feeding (Galibois et al., J. Nutr., 117:2027, 1987). Portal glucose levels reach 9.1 mM, compared with 6.1 to 6.9 mM in peripheral veins (Balks et al., Eur. J. Biochem., 141:645, 1984. The portal vein also carries high concentrations of metabolic regulatory hormones. Insulin (1.8 nM) and glucagon (75 pM) levels are 3 fold and 2.5 fold higher, respectively, than in peripheral veins.

In general, hepatocyte cultures are used to determine the albumin and urea synthesis rates. These rates are measured for cells growing in normal cell culture media, and compared to cells growing in plasma, with and without a plasma supplement added. The cells are first cultured for a week in normal cell culture medium, and then on a later day, e.g., the 5^{th}, 6^{th}, or 7th day, the culture medium is removed, and the cells are grown only in human plasma. Thereafter, the hepatocytes are observed for morphology and viability, and plasma is removed from the culture vessels to allow albumin and urea concentrations to be measured. Standard assays can be used to measure the albumin and urea concentrations.

Another set of important functions expressed by cultured hepatocytes are catalyzed by the cytochrome P450 system of enzymes, which is responsible for the conversion of toxic lipid soluble substances into water-soluble forms that can be excreted from the body. These substances may be of foreign origin (e.g., phenobarbital and 3-methylcholanthrene) or may originate from normal metabolism (e.g., steroid hormones such as testosterone). To assess these functions in hepatocytes during plasma exposure, a toxic substance (e.g., phenobarbital or 3-methylcholanthrene) is added at a low dose to the plasma for a minimum of two days, which induces production of the specialized P450 enzymes that can degrade the toxic compound. The toxic compound is then replaced by an analog that can be converted to a fluorescent product by the same P450 enzymes. The rate of increase in fluorescence then corresponds to the rate of P450 enzymes activity.

The morphological appearance and viability of cultured hepatocytes can be assessed using standard techniques. In general, viable, differentiated hepatocytes in standard culture medium have a typical polygonal morphology, distinct nuclei, and bright cell-cell boundaries indicating the presence of bile canaliculi. In addition, viable cells in a phase contrast image will exhibit calcein fluorescence and little ethidium homodimer-1 staining. Some non-specific fluorescence in a ethidium homodimer-1 image might originate from cell debris trapped in the collagen gel, but this is largely from the non-viable cells included at the time of seeding.

Hepatocytes exposed to plasma without supplementation will exhibit a drastically altered morphology. Cells become circular with less pronounced nuclei, and the bile canaliculi disappear. There is no calcein staining of these cells, while the cell nuclei are clearly stained with ethidium homodimer-1, indicating that massive cell death occurs in hepatocyte cultures exposed to unsupplemented citrated plasma.

As a result of the studies described herein, it is clear that the new plasma supplement greatly enhances the viability and function of hepatocytes, and should therefore be added to extracorporeal plasma circuits entering the cell component in bioartificial liver assist systems during patient treatment.

The invention will be further described in the following examples, which do not limit the scope of the invention described in the claims.

### EXAMPLES

The examples below demonstrate that supplementation with calcium, magnesium, amino acids, and glucagon are essential to maintain high urea synthesis, whereas hydrocortisone and insulin are required for maintenance of a high albumin synthesis rate. During bioartificial liver treatment, these components can be easily infused into the extracorporeal circuit upstream of the cells. As an additional measure, the dose of citrate could be managed to adjust free calcium and magnesium levels to a range low enough to inhibit coagulation, but high enough to maintain hepatocyte activity. Of course, it may be impractical to adjust the citrate dosage in a clinical setting.

### Example 1

### Hepatocyte Isolation and Culture

In this and the following examples, multiwell culture plates (6 well, 35 mm) were purchased from Beckton Dickinson (Franklin Lakes, N.J.). Dulbecco's Modified Eagle Medium (DMEM, with 25 mM glucose and 4 mM glutamine), phosphate buffered saline (PBS), fetal bovine serum (FBS), penicillin and streptomycin, Minimum Essential Medium vitamin mixture, Trypan blue solution, and phenol red solution were purchased from Life Technologies (Gaithersburg, Md.). Insulin was from Novo Nordisk A/S (Bagsuaerd, Denmark), glucagon was from Eli Lilly and Co. (Indianapolis, Ind.), epidermal growth factor (EGF) was from Collaborative Biomedical Products (Bedford, Mass.), and hydrocortisone was from Abbott Laboratories (North Chicago, Ill.). Basal Medium Eagle (BME) amino acid concentrate and powders of serine, glycine, glutamine, and inorganic salts were purchased from Sigma Chemicals (St. Louis, Mo.). Calcein acetoxymethyl ester (calcein-AM) and ethidium homodimer-1 were from Molecular Probes (Eugene, Oreg.).

Hepatocytes were isolated from adult female Lewis rats (Charles River Laboratories) weighing 75-125 g, according to the two-step collagenase perfusion technique described by Seglen (Methods Biol., 13:29, 1976) as modified by Dunn et al., Biotechnol. Prog., 7:237 (1991). Each isolation yielded 2-3×10⁸ hepatocytes. Viability ranged from 85 to 98% as determined by trypan blue exclusion.

Type I collagen was prepared from Lewis rat tail tendons as previously described in Dunn et al. (supra). A collagen gelling solution was prepared by mixing 9 parts of a 1.11 mg/ml collagen solution in 1 mM HCl with 1 part of concentrated (10X) DMEM and kept at 4° C. Six-well culture plates were coated with 0.5 ml of collagen gelling solution and the coated plates were incubated at 37° C. for 1 hour to allow for gelation. The hepatocytes were suspended in culture medium and seeded at a density of 1.0×10⁶ cell per well. The standard hepatocyte culture medium consisted of DMEM supplemented with 10% FBS, 0.5 U/ml insulin, 14 ng/ml glucagon, 20 ng/ml EGF, 7.5 μg/ml hydrocortisone, 200 U/ml penicillin, and 200 μg/ml streptomycin. After incubating at 37° C. in a humidified gas mixture of 90% air/10% CO₂ for 24 hours, the culture medium was removed and the cells were overlaid with a second 0.5 ml layer of collagen gelling solution to form the collagen sandwich. After gelling for 45 minutes at 37° C., 1 ml of fresh medium was added. Fresh medium (1 ml) was supplied to the cultures daily after removal of the previous day's medium. The hepatocytes were cultured for 6 days in standard culture medium prior to plasma exposure.

### Example 2

### Effect of Plasma Supplement on Hepatocytes in Plasma

Expired plasma from a blood bank was pooled, aliquoted, and kept frozen until used for experiments. Plasma supplements were prepared in concentrated form and added individually to plasma as described in the following examples. The final composition of plasma supplemented with the new plasma supplement is listed in Table 3 below. The minimum effective plasma supplement would reduce the hormone levels to the minimums shown in Table 2 above. Vitamins can be added to the supplement when needed by the patient, but they have no effect on hepatocyte viability or function.

TABLE 3
Supplements Added to Human Plasma

| Component | Final | | Volume |
|---|---|---|---|
| (concentration of stock solution) | Concentration* | | Added (mL) |
| Plasma | — | | 79.5 |
| Calcium chloride (1 M) | 1.8 | mM | 0.18† |
| Magnesium sulfate (1 M) | 0.8 | mM | 0.08† |
| Sodium monophosphate (1 M) | 0.9 | mM | 0.09 |
| Sodium bicarbonate (0.88 M) | 44 | mM | 5 |
| Penicillin (20,000 U/mL) and | 200 | U/mL | 1 |
| streptomycin (20 mg/mL) | and | | |
| | 200 | μg/mL | |
| Insulin (100 U/mL) | 500 | mU/mL | 0.5 |
| Glucagon(10 μg/mL) | 14 | ng/mL | 0.14 |
| Hydrocortisone (2 mg/mL) | 7.5 | μg/mL | 0.375 |
| Glycine (0.8 M) | 0.4 | mM | 0.05 |
| L-serine (0.8 M) | 0.4 | mM | 0.05 |
| L-glutamine (0.2 M) | 4.0 | mM | 2 |

| | | |
|---|---|---|
| Basal Medium Eagle amino acids | — | 8 |

| | | |
|---|---|---|
| L-arginine (5 mM) | 0.4 | mM |
| L-cystine (2.5 mM) | 0.2 | mM |
| L-histidine (2.6 mM) | 0.2 | mM |
| L-isoleucine (10 mM) | 0.8 | mM |
| L-leucine (10 mM) | 0.8 | mM |
| L-lysine (10 mM) | 0.8 | mM |
| L-methionine (2.5 mM) | 0.2 | mM |
| L-phenylalanine (5 mM) | 0.4 | mM |
| L-threonine (10 mM) | 0.8 | mM |
| L-tryptophan (1 mM) | 0.08 | mM |
| L-tyrosine (5 mM) | 0.4 | mM |
| L-valine (10 mM) | 0.8 | mM |

*Values shown are the concentrations of the individual supplements in the final plasma mixture excluding what was already present in the plasma.
†Needed only when supplementing citrated plasma.

The sodium monophosphate and the sodium bicarbonate are buffers. The pH of the mixture should be about 7.4, which is physiological pH. Without these salts, the pH would likely be too acidic, which may cause precipitation of some of the amino acids ad have an adverse effect on the cells.

In each set of experiments, components of the supplementation to be included or excluded were designated according to the result from prior experiments. On the 7th day after isolation, the culture medium was removed and hepatocytes were fed with 1 ml of plasma. Cells were fed with fresh plasma daily thereafter. Control cultures were maintained in hepatocyte culture medium throughout. For the hormone study only, the 6-day-old collagen double-gel cultures were washed 10 times with unsupplemented DMEM to remove residual hormones in the collagen gel, and twice with medium or plasma identical to the following experimental condition.

The morphology of hepatocytes cultured in the collagen gel sandwich configuration was examined throughout the course of the 7-day plasma exposure. For viability assessment, cultures were washed three times with PBS and incubated for 30 min in 1 ml PBS with 5 μM calcein-AM and 10 μM ethidium homodimer-1. Intracellular conversion of non-fluorescent calcein-AM to intensely fluorescent calcein and ethidium homodimer-1 binding nucleic acid were visualized on a fluorescent microscope equipped with standard fluoroscein and rhodamine filter sets. Digital photomicrographs were captured using a CCD V1470 camera (Optitronics, Goleta, Calif.) mounted on a Nikon Diaphot inverted microscope (Nikon, Tokyo, Japan).

Culture supernatants were collected daily after a 24 hour culture period. Albumin concentration in standard culture medium and in plasma was determined by an enzyme-linked immunosorbent assay as described in Dunn et al. (1991, supra) utilizing a polyclonal antibody to rat albumin (Cappel Laboratories, Aurora, Ohio). A standard curve was derived using background culture medium prepared by incubation with an empty collagen gel for the same period as the cell culture, and chromatographically purified rat albumin (Cappel Laboratories). Urea concentration in standard culture medium and in plasma was measured by the specific reaction of diacetyl monoxime with urea (Blood Urea Nitrogen Assay Kit, Sigma Diagnostics).

Rates of secretion were normalized to the number of cells initially seeded. Results are presented as an average±standard deviation (S. D.) of three independent experiments, with each experiment employing three replicates. The statistical significance of observed differences was determined by two-way analysis of variance and post hoc Tukey's test. An alpha value of 5% was considered significant.

### Example 3

### Effect of Sodium on Hepatocyte Function

Normal hepatocyte culture medium is high in sodium. Thus, simply adding concentrated medium to plasma results in a high level of sodium, which is not useful for clinical applications because of the resulting high osmolarity, which could be harmful to blood cells and possibly the patient. As a result, we tested whether sodium chloride could be eliminated to reduce the osmolarity of supplemented plasma. Sodium chloride in the supplementation was titrated between 0 mM and 110 mM, the latter being the level used in standard culture medium. These levels indicate the increase in molarity of the plasma after supplementation.

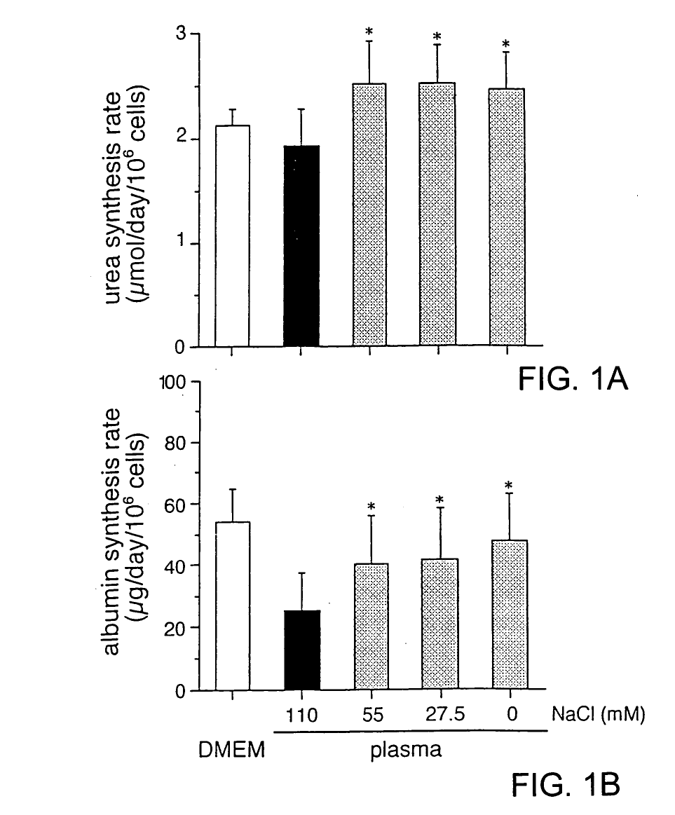

FIGS. 1A and 1B are graphs showing the effect of sodium chloride concentration in supplemented plasma. Rat hepatocytes were cultured in the collagen sandwich configuration and in standard culture medium for 6 days prior to plasma exposure. The cells were then switched to citrated human plasma supplemented with 5× concentrated standard culture medium excluding sodium chloride, which was separately added at various fractions of the level found in standard culture medium. Control cultures (DMEM) remained in standard culture medium throughout. Urea (FIG. 1A) and albumin (FIG. 1B) synthesis rates on day 5 of plasma exposure are shown. Each data point is the average±S. D. of three separate experiments each employing triplicate cultures. There was a significant difference between cultures in plasma supplemented with 5×medium concentrate with full (10 mM) sodium chloride and 55 mM or lower NaCl. Synthesis rates for both urea and albumin were increased upon reducing the sodium chloride levels by 50% or more. Therefore, the plasma supplement does not require sodium.

### Example 4

### Effect of Inorganic Salts

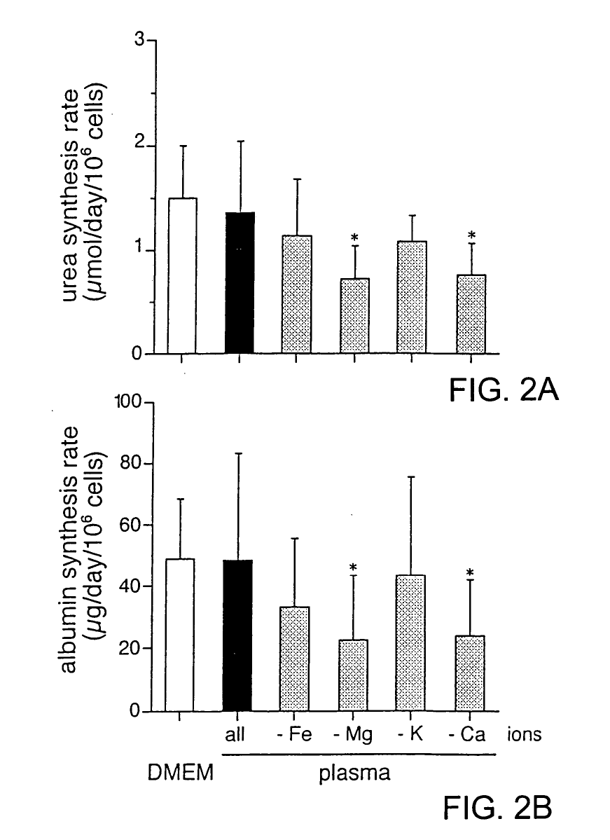

In the following experiment, cells were exposed to plasma supplemented with a 5× culture medium concentrate, with the exception of sodium chloride and either one of the following: ferric nitrate, magnesium sulfate, calcium chloride, or potassium-chloride. Rat hepatocytes were cultured in the collagen sandwich configuration and in standard culture medium for 6 days prior to plasma exposure. The cells were then switched to citrated human plasma supplemented with all the components of the 5×culture concentrate except either one of the following: ferric nitrate (—Fe), magnesium sulfate (—Mg), potassium chloride (—K), or calcium chloride (—Ca). Control cultures (DMEM) remained in standard culture medium throughout. FIGS. 2A and 2B show urea and albumin synthesis rates, respectively, on day 5 of plasma exposure. Each data point is the average±S. D. of three separate experiments each employing triplicate cultures.

Compared with cells cultured in plasma supplemented with all four of the above inorganic salts, cells cultured in plasma not supplemented with calcium chloride or magnesium chloride produced significantly less urea and albumin (FIGS. 2A and 2B). However, removal of ferric nitrate or potassium chloride had no significant effect on these hepatocyte functions. The effect of combined removal of sodium chloride, ferric nitrate, and potassium chloride was also investigated. While sodium chloride removal enhanced both urea and albumin synthesis rates, variations in the other two salts (Fe and K) made little difference in albumin and urea synthesis rates. The results are also shown in Table 4 below, in which the values shown are mean values with standard deviation across three independent experiments. Asterisks (*) indicate a significant difference (p<0.05) from standard medium cultures.

TABLE 4
Effect of Inorganic Salt and Glucose
Supplementation on Hepatocyte Function

| | | | | Urea | Albumin |
|---|---|---|---|---|---|
| | | | | synthesis rate* | synthesis rate* |
| | | | | (μmol/day/ | (μmol/day/ |
| NaCl | Fe(NO₃)₃ | KCl | Glucose | 10⁶ cells) | 10⁶ cells) |
| + | + | + | + | 1.93 ± 0.38 | 25.4 ± 11.9 |
| − | + | + | + | 2.45 ± 0.35† | 47.4 ± 15.3† |
| − | − | − | + | 2.35 ± 0.35† | 45.0 ± 17.1† |
| − | − | − | − | 2.48 ± 0.46† | 40.6 ± 14.7† |

| | | |
|---|---|---|
| Control in standard culture | 2.12 ± 0.15 | 53.9 ± 10.5† |
| medium | | |

*average ± S.D. (n = 9)
†Significantly different from cultures in plasma supplemented with all the components of standard culture medium
Thus, the plasma supplement should include Ca and Mg salts, but no Fe, Na, or K salts.

### Example 5

### Effect of Glucose

The glucose level measured in pooled plasma was approximately 24 mM, which is sufficient to support hepatocytes. In addition, as shown in Table 4 above, tests indicated that elimination of glucose from the plasma supplement did not affect either albumin or urea synthesis rates. Thus, the plasma supplement need not include glucose.

### Example 6

### Effect of Amino Acids

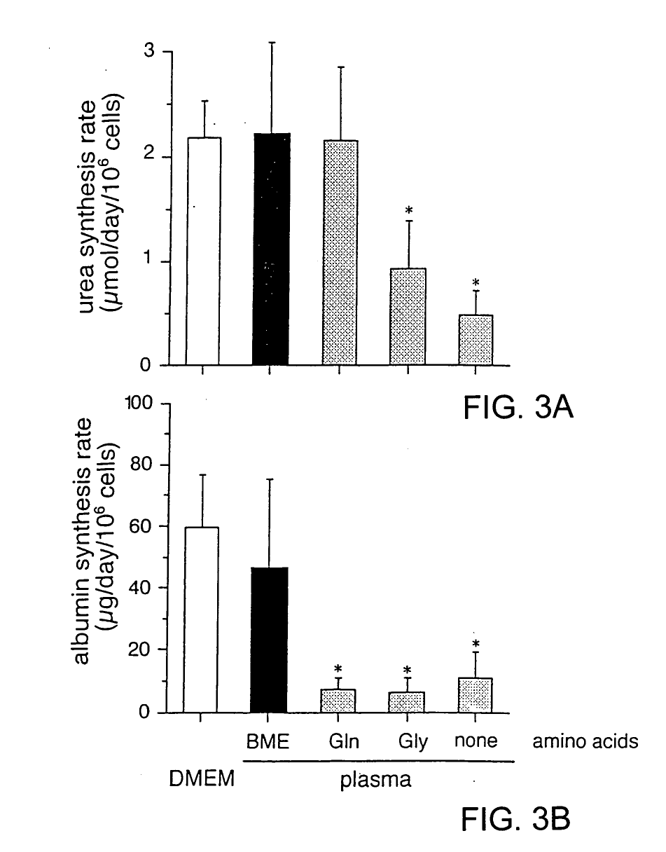

The following experiments were used to assess the role of amino acid supplementation in the maintenance of hepatocyte-specific functions. In addition, tests were done to determine whether multiple amino acids can be replaced with a single amino acid without decreasing cell-specific functions. Plasma was supplemented with a 5-fold concentrate of the standard culture medium from which sodium chloride, ferric nitrate, potassium chloride, and glucose were excluded. The amino acid supplementation consisted of four-fold Basal Medium Eagle (BME) amino acids (total concentration in the plasma after supplementation of 10.1 mM, including 4 mM glutamine), 10 mM glutamine (Gln), 10 mM glycine (Gly), or no amino acids (none). Control cultures (DMEM) remained in standard culture medium throughout. Urea and albumin synthesis rates on day 5 are shown in FIGS. 3A and 3B.

Rat hepatocytes were cultured in the collagen sandwich configuration and in standard culture medium for 6 days prior to plasma exposure. The cells were then switched to citrated human plasma supplemented with 5× concentrated standard culture medium as described above, with the amino acid component consisting of either Urea (3A) and albumin (3B) synthesis rates on day 5 of plasma exposure are shown. Each data point is the average±S. D. of three separate experiments each employing triplicate cultures.

As shown in FIG. 3A, the urea synthesis rate was maintained in the glutamine-only-supplemented group, but decreased by 43% in the glycine-only-supplemented group. Urea synthesis was lowest in the absence of amino acid supplementation. As shown in FIG. 3B, cells exposed to plasma with 10 mM glutamine only, 10 mM glycine only, or no amino acids synthesized significantly less albumin than those exposed to plasma with BME amino acid supplementation.

Thus, the plasma supplement should include at least glutamine (if only urea synthesis is of concern), and otherwise should include additional amino acids, e.g., the amino acids in the BME amino acid group shown in Tables 1 and 3 herein. Based on the data, using 10 mM glutamine alone supports viability and urea synthesis, but not albumin secretion and most likely not general protein synthesis. All of the amino acids listed in this example are necessary to maintain protein synthesis in hepatocytes.

### Example 7

### Effect of Hormones

The effect of hormone levels on the function and morphology of hepatocytes was investigated. To insure that residual hormones from pre-experimental culture in standard culture medium were removed prior to plasma exposure, repeated washing of the cultures with serum and hormone-free DMEM was carried out.

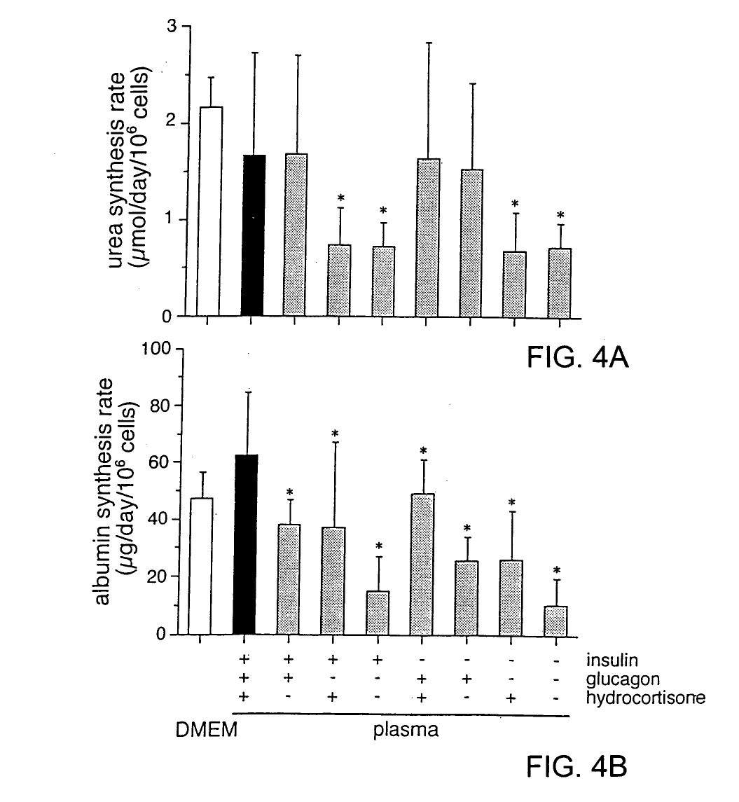

FIGS. 4A and 4B show the synthesis rates of urea and albumin, respectively, after 5 days of plasma exposure. Rat hepatocytes were cultured in the collagen sandwich configuration and in standard culture medium for 6 days prior to plasma exposure. After 12 washes to remove residual hormones in the cultures, the cells were switched to citrated human plasma supplemented with 5× concentrated standard culture medium as described in the text, with (+) or without (−) 500 mU/ml insulin, 14 ng/ml glucagon, or 7.5 μg/ml hydrocortisone (in the supplemented plasma—the concentrations in the plasma supplement are 5×greater), as indicated. Control cultures (DMEM) remained in standard culture medium throughout. Each data point is the average±S. D. of three separate experiments each employing triplicate cultures.

As shown in FIG. 4A, urea synthesis decreased significantly in the absence of glucagon supplementation irrespective of the presence of insulin and hydrocortisone. On the other hand, as shown in FIG. 4B, albumin synthesis was sensitive to all three hormones, as it was significantly reduced by removal of glucagon, hydrocortisone, or insulin alone. Furthermore, the effect of simultaneously removing 2 or 3 hormones appeared to be additive. As a result, the plasma supplement should preferably include all three hormones, insulin, glucagon, and hydrocortisone.

### Example 8

### Effect of Plasma Supplement on Hepatocytes in Heparinized Plasma

Heparin-treated human plasma was purchased from Rockland Immunochemicals, Inc. (Gilbertsville, Pa.). A plasma solution was prepared by mixing plasma and the plasma supplement at a ratio of 87:13. Six-day old sandwiched hepatocyte cultures were exposed to one of four conditions for seven days: (1) standard culture medium, (2) heparin-treated plasma with no supplementation, (3) plasma supplemented with hydrocortisone (7.5 μg/ml) and insulin (50 μU/ml), together with vitamins, phenol red, antibiotics (Life Technologies), sodium monophosphate, and sodium bicarbonate (Sigma Chemicals, St. Louis, Mo.) at the same concentration as in the standard culture medium, or (4) plasma supplemented with everything listed in (3) plus a four-fold concentrate of Basal Medium Eagle amino acids (Sigma). The composition of the amino acids in the supplementation was essentially as shown in Table 1 above.

On the first day of plasma exposure, medium in the collagen gel was replaced with plasma as follows: (1) culture medium was removed followed by addition of 1 ml of plasma solution; (2) cells were incubated for 2 hours during which time the liquid in the collagen gel equilibrated with the liquid above; (3) the liquid above the gel was then removed and fresh plasma was added. This whole process was repeated two additional times. Thereafter, 1 ml of plasma solution or standard culture medium was added daily to the collagen sandwich cell culture, after removal and collection of the previous day's plasma or standard culture medium.

The microscopic appearance of the cultured hepatocytes was monitored daily throughout the one-week period. Cells cultured in standard culture medium retained typical features of hepatocytes, including a polygonal structure and bile canalicular formation, until day 7 of the experiment. Cells were cultured for 6 days prior to experiments to establish stable hepato-specific functional levels. At day 1 of plasma exposure (or day 7 of culture), the cells exposed to heparin-treated plasma without amino acid or hormone supplementation began to exhibit lipid accumulation. On day 7 of plasma exposure, the cytoplasm contained lipid vesicles of varying sizes. The nuclei became enlarged and the refractile bile canaliculi became less pronounced. In some cells, the nucleus was totally obscured by engorged lipid droplets.

Hepatocytes cultured in heparinized plasma with hormone supplementation revealed similar morphologic characteristics as in the standard culture medium cultures following the one-week exposure. The nuclei remained intact and bile canaliculi observed were similar to those in standard culture medium cultures. Observed lipid accumulation was quantified as shown in Table 5. With hormone supplementation, triglyceride content was similar to that in medium, with or without amino acid supplementation. Cells exposed to'non-supplemented plasma contained significantly higher amount of triglyceride. Taken together, hydrocortisone and insulin supplementation elicited a beneficial effect in maintaining hepatocyte morphology in plasma.

TABLE 5
Triglyceride Content in Cells after 7-Day Plasma Exposure

| | Intracellular Triglyceride |
|---|---|
| Treatment | (ng/μg DNA) |
| Standard Culture Medium | 81.0 ± 56.1 |
| Plasma | 294.1 ± 236.2* |
| Plasma + Hormones | 48.3 ± 14.9 |
| Plasma + Hormones + Amino Acids | 58.2 ± 29.9 |

Value shown are mean values with standard deviation across three independent experiments.
Asterisk (*) indicates a significant difference (p < 0.05) from standard medium cultures.

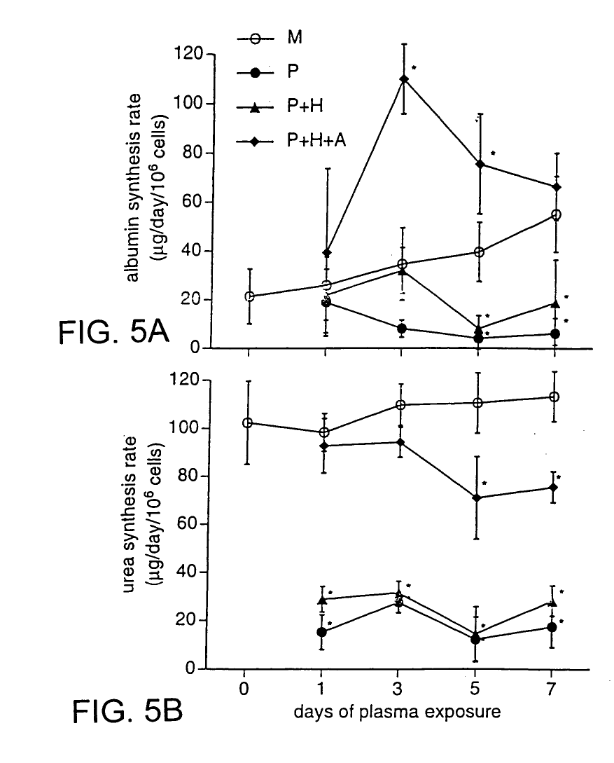

Albumin synthesis and urea synthesis were also measured. Albumin synthesis rate for cells cultured in standard culture medium (M, o) gradually increased (FIG. 5A), whereas urea synthesis rate remained relatively constant, with small variations over the course of the experiment (FIG. 5B).

As shown in FIG. 5A, albumin synthesis rate for the hepatocytes exposed to heparinized human plasma with hormone plus amino acid supplementation (P+H+A, ♦) peaked on day 3 of plasma exposure and remained higher or similar to that in standard culture medium (M, o). On the other hand, for cells exposed to plasma supplemented with hormones only (P+H, ▴), albumin synthesis rate declined after day 3, reaching a significantly lower level compared with cells cultured in standard culture medium after day 5. Likewise, with no supplementation, albumin synthesis rate declined after day I (P, ●). The albumin synthesis rate for cells cultured in plasma without amino acid supplementation was significantly lower than that observed in plasma with amino acid plus hormone supplementation from days 3 to 7. The addition of hormones without amino acid supplementation did not change this result.

As shown in FIG. 5B, the urea synthesis rate for cells exposed to plasma with amino acid plus hormone supplementation (P+H+A, ♦) was similar to the rate for those in standard culture medium (M, o) until day 3. Although they were lower than those in standard culture medium after day 5, they were significantly higher than those for cells exposed to plasma without amino acid supplementation. On the other hand, cells exposed to plasma without amino acid supplementation, with (P+H, ▴) or without hormones (P, ●), exhibited low levels of urea synthesis from the first day of plasma exposure onwards. The observed rates were significantly lower than that for cells cultured in standard culture medium or heparinized human plasma supplemented with amino acids and hormones.

Based on these results, it is clear that plasma supplemented with hydrocortisone and insulin can maintain hepatocyte morphology for at least one week in a liver assist system. Furthermore, amino acid supplementation prevents a decrease in albumin and urea synthesis, both of which are essential hepatocyte functions.

### Example 9

### Co-Culture of Hepatocytes and Fibroblasts in Plasma

The following experiments were carried out to demonstrate the usefulness of the plasma supplement in enhancing liver-specific functions of hepatocytes co-cultured with fibroblasts. Hepatocytes were seeded in collagen-coated 6-well plates at a density of 5×10⁴ cells/well. After incubating at 37° C. in a humidified gas mixture of 90% air/10% CO₂ for 24 hours, the culture medium was removed and 1.5×10⁵ 3T3-J2 cells (mouse embryonic fibroblasts) per well were seeded to create hepatocyte-3T3 co-cultures. The co-cultures were maintained in 0.4 ml standard hepatocyte culture medium, which was replaced daily. The 3T3-J2 cell line was from American Type Culture Collection (Rockville, Md.) and used within 12 passages.

Starting on the 6th day of culture, cells were fed with plasma, which was changed daily thereafter. Control cultures remained in standard hepatocyte culture medium. For cytochrome P450 induction, 3-methylcholanthrene or phenobarbital were added to the culture medium or plasma at a final concentration of 2 μM and 1 mM, respectively, before adding to the cells.

In some experiments, plasma was supplemented with 8% v/v 50× BME amino acid solution, 2% v/v 200 mM glutamine solution, 0.5 U/ml insulin, 14 ng/ml glucagon, and 7.5 μg/ml hydrocortisone. Control unsupplemented plasma was prepared by dilution with a similar volume of isotonic saline.

Cytochrome P450 activity induced by 3-methylcholanthrene was measured as ethoxy-resorufin o-dealkylase (EROD) activity, and that induced by phenobarbital as pentoxy-resorufin o-dealkylase (PROD) activity. EROD and PROD activities were determined using an in situ assay described previously in the literature (see, e.g., Behnia et al., Tissue Eng., (in press) 2000), and Donato et al., Anal. Biochem., 213:29, 1999). Briefly, the incubation mixture contained 3.3 μM resorufin-derived substrate (ethoxy- or pentoxy-resorufin) and 60 μM dicumarol in Hank's buffered salt solution with 20 mM, HEPES. Dicumarol was used to prevent the disappearance of the fluorescent resorufin product due to further metabolism by cytosolic oxidoreductases (Luber et al., Mat. Res., 142:127, 1985). To perform the assay, culture medium was removed, the cells were washed twice with 0.5 ml of HBSS, and the pre-warmed incubation mixture was added. The plates were immediately placed into a microplate fluorometer (Fmax; Molecular Devices, Sunnyvale, Calif.) set at an incubation temperature of 37° C. The increase in fluorescence intensity was recorded using excitation/emission filters of 530/590 μm, respectively, at 2 minute intervals for 8 minute (EROD activity), or at 2.5 minute intervals for 15 minute (PROD activity). Resorufin concentrations were calculated based on known resorufin standards (0-5 μM). The cells were washed twice with HBSS following cytochrome P450 measurements and returned to the incubator following addition of medium or plasma. Albumin and urea synthesis were also measured.

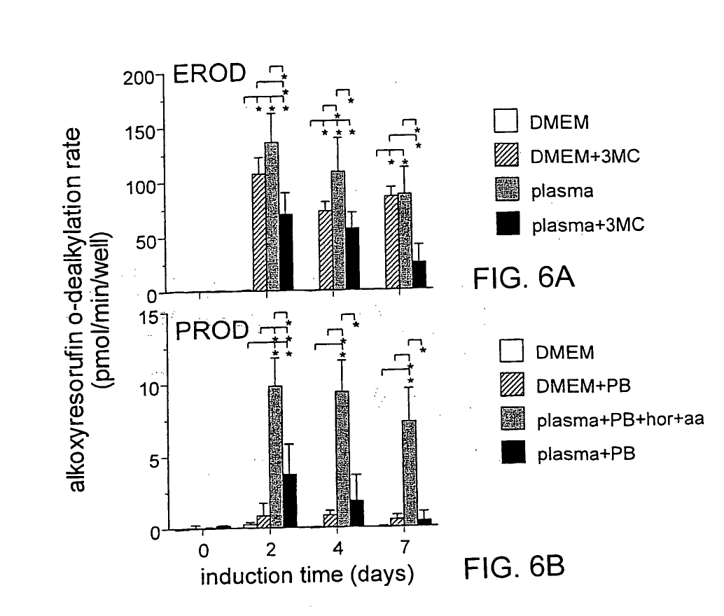

Cytochrome P450 activities of cells cultured in plasma with or without supplementation were tested in parallel for comparison. In these experiments, cells exposed to standard hepatocyte culture medium without inducer were used as negative controls, and cells exposed to medium with inducer were used as positive controls. The negative controls did not show any measurable cytochrome P450 activity, as expected. The activities in EROD and PROD activities induced in unsupplemented plasma compared favorably, and, at times, even exceeded that measured in the cells exposed to medium with inducers. However, cytochrome P450 activities in cells exposed to unsupplemented plasma all decreased between the 2nd and 7th day of induction (FIGS. 6A and 6B). In contrast, with amino acid and hormonal supplementation, no significant decrease in EROD or PROD activities between days 2 and 7 was observed. Furthermore, both cytochrome P450 activities in supplemented plasma significantly exceeded that measured in unsupplemented plasma at all times examined.

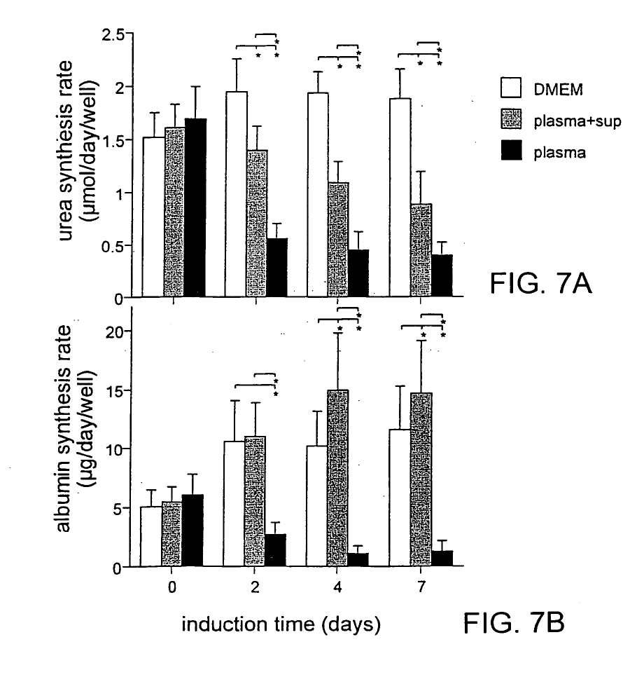

Other liver-specific functions of the hepatocyte co-cultures were monitored during the 7-day exposure to plasma. We observed a stable expression of urea and albumin in the hepatocyte co-cultures (FIGS. 7A and 7B). In plasma supplemented with amino acids, insulin, glucagon, and hydrocortisone, hepatocytes maintained urea synthesis rates between 47 and 72% of that observed in control cultures in medium on the same days (7A), whereas albumin synthesis rates were 104 to 127% of the controls (7B). Without these supplements, both urea and albumin synthesis rates declined rapidly.

### Example 10

### Clinical Use

The liver assist system consists of an extracorporeal device connected to the patient via the circulatory system. A plasma separator can be used as an interface between the patient and the device to prevent immune cells from entering the device. The plasma supplement is added continuously to the plasma before the latter enters the bioartificial liver assist device. A plasma supplement in sterile water as described in Table 3 is used. The flow rate of the supplement is adjusted so that 1 part of supplement is mixed with 4 parts of plasma. If whole blood is used instead of plasma, the plasma supplement is continuously added to the blood entering the device. Plasma or blood levels of metabolites are monitored every 4-6 hours and the amount of plasma supplement is adjusted accordingly so that metabolites in the body fluid entering the device remain in the useful range.

Frequent monitoring is useful to assess the progression of hepatic disease and effectiveness of bioartificial liver treatment. Once the patient's metabolite levels returned to the normal range, bioartificial liver treatment can be discontinued.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A plasma or blood supplement to be mixed with plasma or blood, the supplement comprising amounts of insulin, glycogen, hydrocortisone, and one or more amino acids sufficient to provide a concentration in plasma or blood of:
from 50 μU/mL to 1000 mU/mL insulin;
from 0.7 ng/mL to 28 ng/mL glucagon;
from 75 ng/mL to 15 μg/mL hydrocortisone; and
from 5.3 to 27.6 mM of one or more amino acids.

2. The supplement of claim 1, wherein the supplement provides a concentration in plasma or blood of 2.0 mM-10.0 mM of L-glutamine.

3. The supplement of claim 2, wherein the supplement further provides a concentration in plasma or blood of:
0.1 mM-0.8 mM of L-histidine,
0.4 mM-2.0 mM of L-isoleucine,
0.4 mM-2.0 mM of L-leucine,
0.4 mM-2.0 mM of L-lysine,
0.1 mM-0.8 mM of L-methionine,
0.2 mM-1.0 mM of L-phenylalanine,
0.4 mM-2.0 mM of L-threonine,
0.04 mM-0.2 mM of L-tryptophan, and
0.4 mM-2.0 mM of L-valine.

4. The supplement of claim 3, wherein the supplement further provides a concentration in plasma or blood of:
0.2 mM-1.0 mM of Glycine,
0.2 mM-1.0 mM of L-serine,
0.2 mM-1.0 mM of L-arginine,
0.1 mM-0.8 mM of L-cystine, and
0.2 mM-1.0 mM of L-tyrosine.

5. The supplement of claim 1, further comprising a calcium salt in an amount sufficient to provide a concentration in plasma or blood of from 1.8 to 12.8 mM of the calcium salt.

6. The supplement of claim 5, wherein the calcium salt is calcium chloride.

7. The supplement of claim 1, further comprising a magnesium salt in an amount sufficient to provide a concentration in plasma or blood of from 0.8 to 5.7 mM of the magnesium salt.

8. The supplement of claim 7, wherein the magnesium salt is magnesium sulfate.

9. The supplement of claim 1, wherein the supplement provides a concentration in plasma or blood of 100 μU/mL to 500 mU/mL insulin.

10. The supplement of claim 1, wherein the supplement provides a concentration in plasma or blood of 1.4 ng/mL to 14 ng/mL of glucagon.

11. The supplement of claim 1, wherein the supplement provides a concentration in plasma or blood of 150 ng/mL to 7.5 μg/mL of hydrocortisone.

12. The supplement of claim 2, wherein the supplement provides a concentration in plasma or blood of:
4.0 mM-10.0 mM of glutamine,
0.2 mM-0.8 mM of L-histidine,
0.8 mM-2.0 mM of L-isoleucine,
0.8 mM-2.0 mM of L-leucine,
0.8 mM-2.0 mM of L-lysine,
0.2 mM-0.8 mM of L-methionine,
0.4 mM-1.0 mM of L-phenylalanine,
0.8 mM-2.0 mM of L-threonine,
0.08 mM-0.2 mM of L-tryptophan, and
0.8 mM-2.0 mM of L-valine.

13. A method of enhancing hepatocyte viability and function in a liver assist system (LAS) during clinical use, the method comprising
culturing hepatocytes in the LAS using standard culture medium;
after the hepatocytes are established in the LAS, removing the culture medium and adding plasma or blood; and
adding to the LAS the supplement of claim 1 in an amount effective to enhance the viability and function of the hepatocytes.

14. A composition comprising plasma or blood and the supplement of claim 1.

15. The composition of claim 14, wherein the plasma or blood is human plasma or blood.

16. The composition of claim 14, further comprising citrate in an amount effective to avoid coagulation of blood.

17. The composition of claim 14, further comprising heparin in an amount effective to avoid coagulation of blood.
